(19) United States
(12) Patent Application Publication
(10) Pub. No.: US 2008/0286378 A1
(43) Pub. Date: Nov. 20, 2008

(54) Use of Amniotic Fluid (Af) in Treating Ocular Disease and Injury

(76) Inventors: Ashley Behrens, Sparks Glencoe, MD (US); Beatriz Brito, Caracas (VE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/816,682

(22) PCT Filed: Feb. 21, 2006
(86) PCT No.: PCT/US2006/005986

§ 371 Date: Jul. 2, 2008

Related U.S. Application Data
(60) Provisional application No. 60/655,203, filed on Feb. 22, 2005.

(51) Int. Cl. A61K 35/50; A61P 27/02

(52) U.S. Cl. 424/528

(57) ABSTRACT

Compositions and methods for the treatment of ocular disease and injury are provided. The methods involve the administration of amniotic fluid directly to the eye, for example, as eye drops. The types of diseases and injuries that can be treated in this manner include chemical burns, dry eye and corneal neovascular disorders, corneal opacities (including corneal haze) and inflammatory diseases of the eye.

### BACKGROUND OF THE INVENTION

1. Field of the Invention

The invention generally relates to the treatment of ocular disease and injury (e.g. dry eye and chemical burns). In particular, the invention provides for the treatment of ocular disease and injury by the application of amniotic fluid to the eye.

2. Background of the Invention

Good vision contributes greatly to a person's ability to interact with and function in his or her environment. Diseases of and injuries to the eyes can be severely debilitating, and occur in a wide variety of forms. For example, thousands of chemical burns, which are frequently occupational in nature, occur each year in the United States, and the numbers are even higher in countries with lower worker safety standards. Similarly, dry eye, a disease that is related to some autoimmune disorders and to aging in general, afflicts millions of people world-wide.

A variety of attempts have been made to treat such disorders and injuries in the past, but have met with only partial success. For example, a variety of eye drops are known for use in soothing eye irritation or for supplying artificial tears. Human amniotic membrane (HAM) has been successfully used to treat various surface ocular injuries and disorders. However, the use of HAM involves surgical attachment of the membrane to the surface of the eye, and thus requires the skills of a surgeon. Also, this procedure causes impairment of vision during treatment as the amniotic membrane is not transparent. In addition, the benefits of the procedure last only as long as the membrane is in place, so the procedure is not particularly useful for chronic conditions such as dry eye.

International patent application number PCT/US2003/029390 to Ghinelli describes the use of a human amniotic membrane composition for prophylaxis and treatment of diseases and conditions of the eye and skin. However, this methodology involves considerable processing of the membrane prior to use (e.g. lyophilzation, pulverization, and reconstitution). The potency of some vital factors may be lost or attenuated by such processing.

There is an ongoing need for alternative therapies for the treatment of ocular diseases and injuries, particularly low-cost therapies that are readily accessible easy to use.

### SUMMARY OF THE INVENTION

The present invention provides methods and compositions for the treatment of ocular diseases and injuries. The methods comprise the topical administration of amniotic fluid (AF) to the eye, for example, in the form of eyedrops. Topical delivery of AF has the advantage of avoiding the surgical procedure required with HAM. Therefore, nonsurgical ophthalmologists can prescribe and administer the therapy, giving patients greater access to treatment. In fact, the patients themselves can administer the AF. In addition, unlike the surgical application of HAM, repeated topical applications of AF provide a sustained level of beneficial factors. Further, AF may require less processing than HAM and preparations of HAM, decreasing the cost and increasing the accessibility of the therapy.

The invention provides a method for treating a disorder or injury in an eye, and includes the step of administering amniotic fluid free of amniotic membrane particulate matter to said eye in a quantity sufficient to ameliorate symptoms associated with the disorder or injury. The injury may be, for example, a chemical burn. The disorder may be, for example dry eye, a corneal neovascular disorder, surface inflammation, intraocular inflammation or corneal opacity. In one embodiment, the amniotic fluid free of amniotic membrane particulate matter is human amniotic fluid. In one embodiment, the amniotic fluid free of amniotic membrane particulate matter is in the form of eyedrops. Alternatively, the amniotic fluid free of amniotic membrane particulate matter may be released from a collagen contact lens. In yet another embodiment, the amniotic fluid free of amniotic membrane particulate matter may be lyophilized and reconstituted for administration.

The invention further provides a device and medicament combination for treating a disorder or injury to the eye. The device comprises 1) a housing having a reservoir and an orifice for dispensing selected volumes of fluid medicament, the reservoir being operatively connected to the orifice so as to allow the selected volumes to be dispensed through the orifice; and 2) a fluid medicament which is or contains amniotic fluid free of amniotic membrane particulate matter, the amniotic fluid free of amniotic membrane particulate matter being positioned in the reservoir of the housing. The injury that is treated may be a chemical burn. Alternatively, a disorder such as dry eye or a corneal neovascular disorder (or others) may be treated. In one embodiment, the amniotic fluid free of amniotic membrane particulate matter is human amniotic fluid. In some embodiments of the invention, the device dispenses eye drops; in another embodiment, the device dispenses a spray.

In yet another embodiment, the invention provides a device and medicament combination for treating a disorder or injury to the eye. The device and medicament include a housing having a reservoir and an orifice for dispensing selected volumes of fluid medicament, in which the reservoir is operatively connected to the orifice so as to allow the selected volumes to be dispensed through the orifice; and a fluid medicament which is or contains amniotic fluid that has been centrifuged at 1800 rpm positioned in the reservoir of the housing.

### BRIEF DESCRIPTION OF THE DRAWINGS

FIG. 1A-C: A representative mouse from each group, showing the epithelial defect area (darkest areas, stained green in the original) on postoperative days 2 (left) and 4 (right). A, Group 1 (pre-term human AF); B, Group 2 (term human AF); and C, Group 3 (isotonic saline or control group).

FIG. 2: Transformed (arcsin) data and averaged day 2 and day 4 epithelial defect percentages, along with the mean and 95% confidence intervals of the mean.

FIG. 3A-C: A representative mouse from each group, showing the corneal damage on postoperative days 2 (left column), 7 (middle), and 14 (right). A, Group 1 (pre-term human AF); at day 14, arrows show area of opacity within otherwise transparent cornea. White background due to cataract developed during the photo session. B, Group 2 (term human AF); and C, Group 3 (isotonic saline or control group).

FIG. 4: Assessment of ocular burn score using a generalized estimating equation (GEE). The average change in score over time is shown for each treatment group.

FIG. 5A-C: A representative mouse from each group, showing a histological section at 40× magnification of the burned eye (left column), and contralateral, non-burned eye (right column). A, Group 1 (pre-term human AF); B, Group 2 (term human AF); and C, Group 3 (isotonic saline or control group). Note the increased corneal thickness, inflammatory cell infiltrate, and numerous blood vessels (arrow) in the control group when compared to the human AF treated corneas.

### DETAILED DESCRIPTION OF THE PREFERRED EMBODIMENTS OF THE INVENTION

The present invention provides methods and compositions for the treatment of ocular diseases and injuries. The invention is based on the discovery that amniotic fluid (AF) exhibits high efficacy in healing and regenerating eye tissue when topically applied to the surface of the eye at the site of disease of injury.

The meaning of “amniotic fluid” is well-known to those of skill in the art. Briefly, this is the fluid inside the membrane that forms a sac around the embryo and later the fetus, which is in permanent contact with the fetus and the eye during the gestational period. The fetus and the placenta produce the amniotic fluid. In some embodiments of the invention, the AF that is used is human AF. However, those of skill in the art will recognize that AF from other mammalian species may also be successfully utilized, examples of which include but are not limited to horse, rabbit, lamb, cow sheep, primates, etc.

Those of skill in the art are well-acquainted with methods of safely and humanely obtaining samples of AF, and of the need to maintain sterility of the AF during such procedures. Suitable sources, e.g. of human AF, include AF that is obtained from patients who are undergoing amniocentesis, patients who are undergoing a Caesarean section delivery, and patients undergoing normal delivery using a specially designed receptacle to collect the fluid after rupture of membranes. The AF that is utilized in the present invention is also screened after collection to insure that it is not contaminated with communicable disease agents, such as HIV, HTLV, Hepatitis B and C, syphilis, etc.

The AF that is utilized in the present invention is free of amniotic membrane particulate matter, i.e. it has been clarified after collection. In other words, the AF has been subjected to a procedure that removes, for example, cellular debris, such as that which is sloughed from the amniotic membrane, but that retains macromolecules (e.g. proteins, lipids, nucleic acids, sugars, etc.). Those of skill in the art are familiar with techniques for removing particulate matter from biological samples. Examples of such techniques include but are not limited to centrifugation (e.g. at a speed in the range of from about 1000 rpm to about 5000 rpm, and preferably at least about 1800 rpm. In one embodiment of the invention, the AF is amniotic fluid that has the properties of AF from which amniotic membrane particulate matter has been removed by centrifugation at about 1800 rpm.

In addition, the AF that is utilized in the present invention may be further treated, e.g. in order to promote preservation, lengthen shelf life, etc. Such treatments include but are not limited to sterilization (e.g. by gamma-irradiation); cooling, refrigeration and freezing; etc.

In addition, certain substances may be added to the AF, for example, to prevent the growth of microbes (e.g. antifungal, antibacterial or antiviral agents); other agents that also promote healing (e.g. vitamins); or to improve delivery of the AF to the eye or otherwise enhance the technique (e.g. thickeners, salts, various preservatives, colorants, etc.) Such additions may be made, so long as the compounds do not cause irritation of the eye, and do not interfere with the healing action of the AF.

In another aspect of the invention, the AF may be lyophilized (i.e. freeze-dried) and stored, and then reconstituted for use as necessary. Those of skill in the art are well-acquainted with lyophilization techniques. Reconstitution may be carried out with, for example, physiologically compatible saline solutions. In addition, the lyophilized AF may be reconstituted with AF, for example, if is it desired to make the AF more concentrated.

The AF that is used in the methods may be used “full strength” (i.e. undiluted). Alternatively, a diluted form of AF may be administered. For example, compositions may be administered which contain in the range of about 10 to about 90% AF, or in the range of about 20 to about 80% AF, or in the range of about 30 to about 70%, or in the range of about 40 to about 60%, or alternatively about 50% AF in the composition. In the case of a liquid composition, the dilution may be made with any of several suitable diluants that are known to those of skill in the art, for example, physiologically compatible saline solution, balanced saline solution, sodium hyaluronate, methylcellulose, etc. For other formulation, (e.g. ointments) the % AF refers to the percentage of the total composition that is made up of AF, the rest being made up of ingredients that are well-known to those of skill in the art for manufacturing safe, administrable ointment-type preparations (e.g. various biocompatible oils, fillers, gelling agents and the like). In yet other embodiments of the invention, the AF may also be concentrated by removal of water by any of several techniques that are well-known to those of skill in the art, either essentially all water may be removed (e.g. by lyophilzation) or the amount of water may simply be reduced (e.g. by vacuum filtration, etc.). Conditions which can be treated by the methods of the invention include but are not limited to various injuries to the eye such as chemical burns (e.g. alkali or acid burns); burns caused by heat; injury or irritation caused by surgical procedures such as laser surgery, corneal transplant, cataract removal, various transplant procedures; injuries or irritation caused by exposure to noxious substances such as pollutants, hazardous liquids or fumes, smoke, radiation; etc.

Other conditions that may be treated by the methods of the invention include but are not limited to various diseases of the eye, such as those associated with autoimmune diseases and/or aging (e.g. dry eye); infections (such as parasitic, bacterial, fungal and viral infections); corneal opacities of diverse origin; immunologic reactions to corneal transplant surgery; inflammation of the eye, (either surface inflammation or intraocular inflammation).

In yet another embodiment of the invention, AF is used to treat or prevent corneal neovascular disorders. Examples of such disorder include but are not limited to post-chemical burn status, immunologic diseases such a cicatricial pemphigoid and Stevens-Johnson disease, corneal neovascularization after corneal transplantation, and neovascularization post-herpes simplex infection, etc.

Treatment of the eye with AF may cause complete cessation of symptoms associated with the disease, injury or condition being treated. However, those of skill in the art will recognize that treatment of the eye may not always result in a complete cure. Rather, in some cases the symptoms will be ameliorated at least to some extent, compared to an untreated eye, facilitating the performance and improving the outcome of a corneal transplant.

In general, the AF that is utilized in the practice of the present invention will be liquid AF from which particulate matter has been removed, and will be administered topically to the cornea. Administration of liquid, clarified AF to the eye of a patient may be carried out by any of several methods that are well-known to those of skill in the art, examples of which include but are not limited to as eye drops, as a spray, as a rinse, as an ointment, etc. In a preferred embodiment, the AF is administered as eye drops.

In addition, the AF may be administered with a solid carrier such as a contact lens that is permeated with the AF. Such a device may act as a slow-release drug delivery system, and could be comprised of synthetic material or, alternatively, of a biologic and reabsorbable material, for example, a collagen lens.

In yet other embodiments, the AF may be administered within the eye, e.g. by injection. For certain diseases that present with intraocular neovascularization, the delivery of the fluid may be via intravitreal or sub-retinal injection.

In a further aspect of the invention, a device and medicament combination for treating a disorder or injury to the eye is provided. The combination may be in an eye dropper format that includes a housing with a reservoir and an orifice. The reservoir is operatively connected to the orifice so as to allow selected volumes of liquid to be dispensed through the orifice. The combination also includes a fluid medicament which is or contains AF that is free of particulate matter. The AF is positioned (i.e. located within) the reservoir of the housing, and it is the AF that is dispensed through the orifice. Those of skill in the art will recognize that many styles of such liquid dispensing apparatuses exist which may be used in the combination of the invention, some examples of which are illustrated in FIG. 6.

With reference to FIGS. 6A and B, in one possible embodiment, the reservoir is a flexible bottle 10 and the orifice 11 is located at a narrowed portion 12 of flexible bottle 10. Liquid 20 is dispensed from the reservoir by applying pressure to the sides of the bottle, which causes liquid to be expelled from the orifice. The orifice is designed so that roughly equal volumes 21 (drops) of liquid are expelled, particularly when the bottle is inverted as is shown in FIG. 6B.

With reference to FIGS. 6C and D, in another embodiment, the reservoir is a bottle 30 which includes a dropper 31 positioned within bottle 30, orifice 11 being located at a distal end of dropper 31. Liquid 20 is drawn into the dropper by the release of pressure applied to a flexible portion 32 of the dropper. The dropper is then withdrawn from the bottle (FIG. 6D) and roughly equal volumes 21 of liquid are then expelled from the dropper by applying pressure to flexible portion 32.

Those of skill in the art will recognize that such dispenser/liquid combinations are not limited to those depicted in FIGS. 6A-D. For example, the AF may be dispensed in a single use, disposable eye-dropper device such as those typically used for preservative-free eye drops. Further, such dispensers may also include various other elements such as more complex devices for measuring the quantity of liquid that is withdrawn; pumping mechanisms; spray mechanisms; etc.

In general, the quantity of AF that is dispensed from the device is in the range of from about 20 μl to about 60 μl, and preferably in the range of from about 30 μl to about 40 μl, although this amount may vary depending of such factors as the size of the eye, the severity of the condition being treated, the frequency of administration, etc.

The quantity of AF that is delivered to the eye and the frequency of administration of AF to the eye will vary, depending on factors such as the disease or condition being treated, the age and overall health of the patient, and other factors. In general, the quantity of liquid AF that is administered per eye in a single treatment is in the range of from about 20 μl to about 60 μl, and preferably in the range of about 30 μl to about 40 μl.

Likewise, the frequency of administration will also depend on the particular disease or condition being treated, the condition of the particular patient being treated, etc. Generally, the frequency of administration will be in the range of about 4 to about 8 times per day (i.e. about every 2-6 hours). The regimen may also be altered during treatment, e.g. more AF may be administered more frequently at the beginning of treatment, and less may be needed at the same or a lower frequency during ongoing maintenance therapy. Also, either one or both eyes may be treated, using either the same or different treatment protocols, as required to maximize the health and well-being of the patient that is receiving treatment. For some chronic conditions, treatment with AF may continue indefinitely. For other conditions, once an acceptable level of the reversal of symptoms of the disease or condition is observed, treatment may cease. The planning of such treatment regimens is well known to those skilled in the medical field, and is typically carried out or supervised by a skilled practitioner, e.g. a physician or trained medical technician.

### Examples

### Example 1

### Treatment of Ocular Burns

Prior work with human amniotic membrane (HAM) for ocular surface repair has been encouraging. Beneficial effects of HAM for limiting ocular damage following injuries, including chemical burn, have been reported.¹⁻⁵ It is not clear whether benefit is derived from a mechanical (bandage) effect, or from factors present within its collagen meshwork.⁶⁻⁸ Substantial evidence in support of each of these protective and reparative mechanisms indicates that both may contribute, but the relative participation of each is currently unknown.

Human amniotic membrane is a complex biological tissue. Various cytokines (interleukins-IL) are present in HAM and in amniotic fluid: IL-6 and IL-8,⁹ IL-1α, IL-1β, IL-1 receptor antagonist, and IL-10.^{10,11} The last two have been found to provide strong anti-inflammatory activity.^{11,12} An anti-angiogenic protein, the pigment epithelium derived factor (PEDF), has also been found in HAM.¹³ PEDF is anti-angiogenic in animals models of retinal and corneal neovascularization.¹⁴⁻¹⁶ Some activity promoting corneal reepithelialization has also been reported with this molecule.¹⁷ The presence of these and other cytokines and growth factors with known regulatory roles in inflammatory response and wound healing suggests the therapeutic potential of the biologically active, non-structural proteins in HAM.¹

Biological fluids have been used extensively as therapeutic agents in ocular disease. Autologous blood has been applied to conjunctival blebs to avoid excess filtration following glaucoma procedures.^{18,19} Topical autologous serum is reported to be therapeutic for various ocular surface disorders.²⁰⁻²² The rationale for the use of autologous serum is that its protein composition is similar to that of the tear film, and also some beneficial growth factors may be present in it (epithelial growth factor, vitamin A, TGF-beta).²³ In vivo, HAM is bathed with amniotic fluid and both contain potentially therapeutic constituents. Most of the proteins present in HAM are also found in human AF.²⁴ Potential therapeutic indications for HAM and human AF are therefore predicted to be similar. The structural components and mechanical properties of HAM may be the principal determinants in the choice between these therapeutic approaches.

Human AF should be well tolerated by patients. It is in contact with the ocular surface during embryonic development and modulates wound healing in the fetus.²⁵ Lee and Kim report that human AF promotes faster corneal nerve regeneration and recovery of corneal sensitivity following excimer laser ablation in rabbits.²⁶ Some studies suggest that human AF affects scar formation during wound healing.²⁷⁻²⁸ Human AF has been proposed to enhance nerve regeneration in the neurosurgical setting,²⁹ and minimizes fibrosis associated with hand surgery.³⁰ Based on all these observations, our objective was to evaluate the potential of topical human AF to treat ocular alkali burns in a murine model.

### Methods

### Animals

The study protocol was approved by the Johns Hopkins University Animal Care and Use Committee, and the animals were treated in observance of the ARVO Statement for the Use of Animals in Ophthalmic and Vision Research.

Thirty adult female/male C57BL/6 mice (5-18 weeks old) were used in this trial. All mice were free from ocular disease, and fed with a standard caloric diet for their age. The animals were closely monitored prior to all procedures, and until fully recovered.

### Human Amniotic Fluid

After approval of the Institutional Review Board of the Johns Hopkins University, human AF was obtained from patients with low risk pregnancies attending the Department of Gynecology and Obstetrics, Johns Hopkins Hospital. The fluid was obtained at two different time points of the pregnancy, 16-18 weeks (pre-term human AF group) and 36-38 weeks (term human AF group) of gestational age. The pre-term human AF was obtained from samples to be discarded after routine amniocentesis for karyotyping. The term human AF was obtained from samples to be discarded after fetal lung maturity testing in patients near the estimated date of delivery.

Human AF was pooled from four different patients for each of the experimental groups in this study. The human AF was centrifuged at 1,800 rpm for 10 min and the supernatant was preserved at −20° C. until use, approximately one week after it was obtained. The samples were kept frozen until immediately prior to application and then stored at 4° C. in an effort to minimize potential bacterial proliferation in the samples.

### Corneal Alkali Burn

An alkali burn was created in the right eye of each mouse incorporating minor modifications into a well described model.³¹ Mice were anesthetized with an intraperitoneal injection of ketamine/xylazine (45 mg/kg and 4.5 mg/kg, respectively). After instillation of topical anesthesia (one 20 μl drop of proparacaine 0.5%), a 2 μl drop of 0.15 M sodium hydroxide (NaOH) solution was applied in the right eye of each mouse and left on the ocular surface for 40 s. After the standardized exposure to the alkali agent, the eyes were thoroughly washed with 20 ml of isotonic saline solution, in an attempt to remove the residual chemical agent and protein coagulum. A drop of levofloxacin 0.5% (Quixin, Santen Inc., Napa, Calif.) was instilled in each injured eye after the procedure was completed.

Treatment and Experimental Design

The animals were assigned to three different age/sex-matched groups according to treatment administered: pre-term human AF (Group 1), term human AF (Group 2), and topical isotonic saline solution (Group 3). 5 μl eye drops of the respective treatment were applied five times a day during the first week following injury, and then three times a day during the second week after the injury. In addition, topical levofloxacin 0.5% (Quixin) eye drops were also administered three times a day for one week to all groups. For analgesia, subcutaneous buprenorphine (0.1 mg/kg) was administered to each animal every 12 hours for 4 days. At post-injury days 2, 4, 7 and 14, the eyes were photographed using a digital camera (Nikon Coolpix 990, Nikon Inc., Melville, N.Y.) with a 17× macro lens attached.

Each eye was centered in the screen at the minimum focal distance in order to minimize the effects of magnification and standardize photographic procedures for the purpose of quantitative comparison. The lighting conditions of the room were kept constant in all sessions, and the same camera flash setting was used for each photo recording. Three images were taken for each eye and one was selected for grading and assessment by three independent and blinded examiners.

### Epithelial Defect:

Closure of the epithelial defect was monitored on post-injury days 2 and 4, by the instillation of 5 μl of sodium fluorescein 1% (Sigma Aldrich, St. Louis, Mo.) in the affected eye. Excess fluorescein was rinsed away with 1 ml of isotonic saline solution, and digital photography was then taken. All the images were processed and analyzed by a blinded observer to the treatment. The areas of corneal epithelial defect were outlined using digital imaging software (AxioVision, Carl Zeiss, Inc., Thornwood, N.Y.). The pixel values of these two areas were determined, and the corneal epithelial defect was calculated as a percentage of the total corneal area.

### Ocular Burn Assessment:

In order to evaluate and compare the area of injury, we used a modified and semi-quantitative assessment based on Sotozono et al. and the Roper-Hall classification (Table 1).^{32,33} The digital photographs were analyzed by three independent, blinded observers, who previously met to achieve consensus in the assignment of values according to parameters using the proposed classification. Every eye was assigned a clinical score by each observer. The final result for each eye, at a given time point, was the arithmetic mean of the three scores submitted by the observers.

TABLE 1
Modified ocular burn classification*
CORNEAL OPACITY (DENSITY)

| | |
|---|---|
| 0 | No damage |
| 1 | Mild corneal haze, iris details visible |
| 2 | Corneal haze, iris visible, details obscured |
| 3 | Cornea opaque, iris and pupil obscured |

CORNEAL OPACITY (AREA)

| | |
|---|---|
| 0 | No opacity |
| 1 | Opacity covers less than ⅓ of the corneal surface |
| 2 | Opacity covers more than ⅓ and less than ⅔ of the corneal surface |
| 3 | Opacity covers more than ⅔ of the corneal surface |

HYPHEMA

| | |
|---|---|
| 0 | No hyphema or not visible |
| 1 | Hyphema present |

*A number is given for each of the sub-sections above. The total score will be the sum of all numbers, and will range between 0 and 7 points

On postoperative day 14, animals were euthanized using a CO₂ chamber. Both eyes (experimental burn and contralateral control) from the 4 mice with the greatest percentage of observed change between day 2 and 14 were selected for histology, and fixed in 10% buffered formalin solution for 24 hours. They were then immersed and oriented in Tissue-Tek® optimal cutting temperature compound (Ted Pella Inc., Redding, Calif.), flash frozen using 2 methyl butane in dry ice, and sectioned. Seven micron sections were cut, mounted and stained with hematoxylin and eosin according to standard methods. Following staining, each specimen was examined using an inverted microscope with a 40× objective (Axiovert 200M, Carl Zeiss Inc., Thornwood, N.Y.). Digital images of the central cornea were captured using AxioVision Software. Corneal thickness, inflammatory cell infiltrate, and the presence of blood vessels were analyzed.

### Statistical Analysis

Three comparisons were considered in the statistical analysis: group 1 vs. control, group 2 vs. control, and group 1 vs. group 2. A Bonferroni correction was used to account for multiple testing, and utilized a significance level of p=0.017 (0.05/3) to test for differences. For the percent of epithelial defect data, descriptive statistics were expressed as median and inter-quartile range (IQR=25th, 75th percentile). A variance stabilizing transformation was used, in order to provide an approximately normal distribution of the data. For proportions, this transformation is the arcsin [sqrt(x)], where x lies between 0 and 1.³⁴ After transformation, the areas of epithelial defect on days 2 and 4 were averaged for each mouse. T-tests were then performed to observe the differences between treatment groups.

To assess the reliability of the observers' grading system using the ocular burn classification, the intraclass correlation coefficient (ICC) was calculated.³⁵ This correlation coefficient assesses the proportion of the total variability in the readings attributable to the mice: values of ICC higher than 0.7 show sufficient standardization between readers. The average burn score over the three readers was used in all analyses. Descriptive statistics for the ocular burn classification were expressed as a mean and standard deviation, and t-tests for differences in sample means were used to look at differences between treatment groups for day 2 scores; change between day 2 and day 7 scores; and change between day 7 and day 14 scores. The change in score was assessed over time using generalized estimating equations (GEE), assuming a normal probability distribution, and exchangeable correlation structure.³⁶ This model allowed assessment of the change in score over time, and accounts for the correlation between measurements taken from the same mouse. Score was modeled on day, treatment, and day by treatment interaction.

### Results

One mouse from group 2 died from an unknown cause on day 7, after the eye had been photographed. This mouse was not included in any statistical analysis relevant to day 14.

### Epithelial Defect

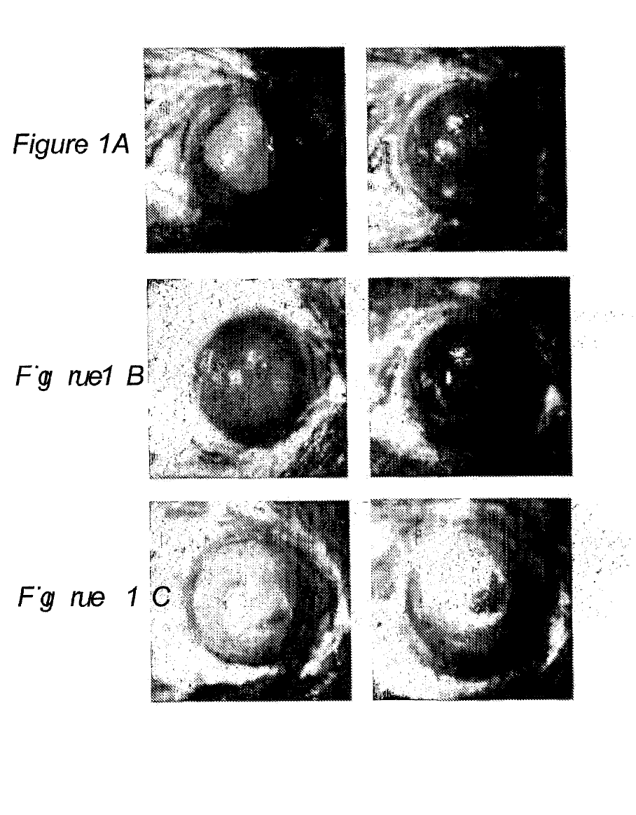

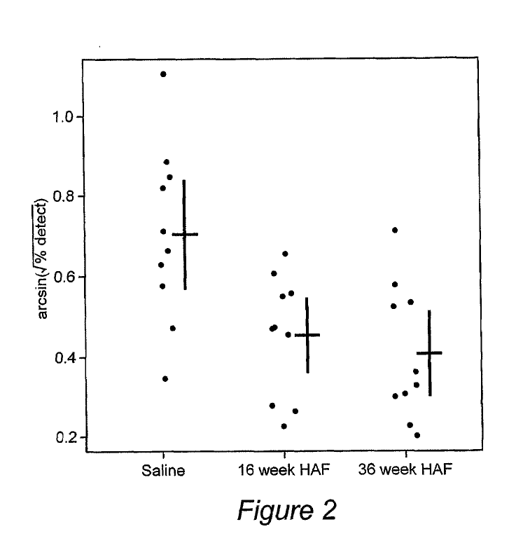

Descriptive statistics are shown in Table 2. The average epithelial defect for days 2 and 4 was significantly smaller in group 1 (p=0.0076) and group 2 (p=0.0031), when compared to the control group. There was no significant difference between the two human AF treated groups (p=0.5279) (FIGS. 1A-C and 2).

TABLE 2
Epithelial defect on days 2 and 4. Descriptive statistics are shown
as median epithelial defect (%) and interquartile range (IQR)

| | Median defect | | Median defect | |
|---|---|---|---|---|
| Group | day 2 | IQR | day 4 | IQR |
| Group 1 | 35.61% | 19.39, 38.57 | 9.92% | 8.57, 11.27 |
| Pre-term | | | | |
| Human | | | | |
| AF | | | | |
| Group 2 | 16.61% | 9.54, 47.42 | 7.30% | 5.96, 8.97 |
| Term | | | | |
| Human | | | | |
| AF | | | | |
| Group 3 | 63.19% | 53.39, 78.71 | 18.91% | 11.71, 27.64 |
| Saline | | | | |

### Ocular Burn

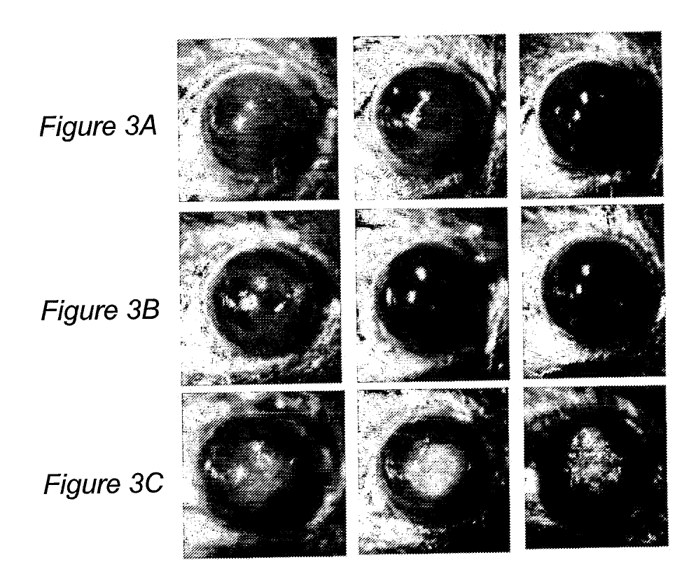

The ICC determined for the ocular burn classification was 0.82. Overall, there was less ocular damage and the corneas were significantly clearer in both human AF groups, as compared to controls (FIG. 3A-C).

Descriptive statistics are presented in Table 3. On day 2 there was no significant difference between groups (group 1 vs. control, p=0.5521; group 2 vs. control, p=0.7102; group 1 vs group 2 p=0.8000). The change in ocular damage from day 2 to day 7 was 0.50 (SD 1.03) for group 1, 0.83 (SD 1.69) for group 2, and −0.83 (SD 1.09) for group 3. This value was significantly greater in both human AF groups when compared to saline (group 1 vs. control p=0.0117, group 2 vs. control p=0.0173). The difference between groups 1 and 2 was not statistically significant (p=0.6012).

TABLE 3
Ocular damage on post-operative days 2, 7 and 14. Score
is presented as mean units and standard deviation (SD).

| | | | | | Mean | |
|---|---|---|---|---|---|---|
| | Mean Score | | Mean score | | score | |
| Group | Day 2 | SD | day 7 | SD | day 14 | SD |
| Group 1 | 3.20 | 1.48 | 2.70 | 1.69 | 2.1 | 1.31 |
| Pre-term | | | | | | |
| Human AF | | | | | | |
| Group 2 | 3.36 | 1.43 | 2.53 | 1.64 | 2.04 | 1.37 |
| Term Human | | | | | | |
| AF | | | | | | |
| Group 3 | 3.63 | 1.71 | 4.46 | 1.99 | 4.13 | 2.32 |
| Saline | | | | | | |

The change in score observed from day 7 to day 14 was not statistically significant in any of the groups. The score change in this period was 0.6 (SD 0.75) for group 1 (p=0.6130 when compared to control), 0.19 (SD 0.45) for group 2 (p=0.7763 when compared to control), and 0.33 (SD 1.46) for the control group.

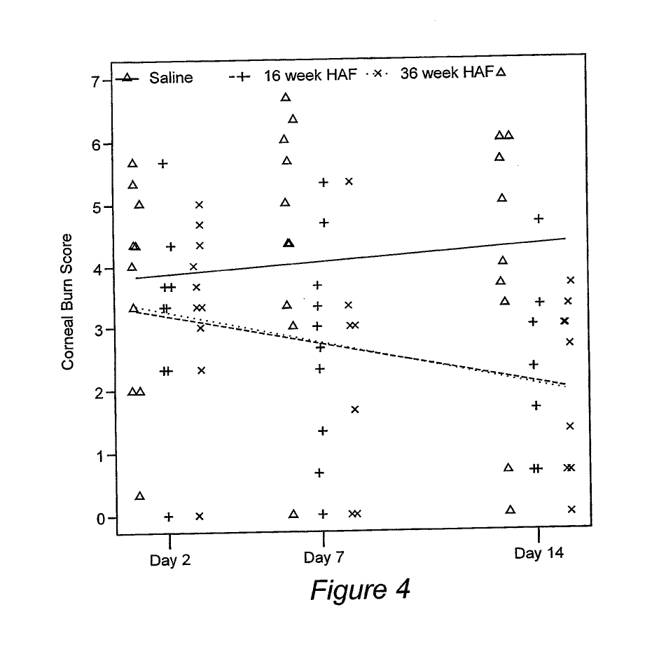

The overall change in burn score between days 2 and 14 was different between group 1 vs. control (difference in slope=−0.127, p=0.009), and between group 2 vs. control (difference in slope=−0.134, p=0.012) (Table 4); and again, there were no differences between group 1 vs. group 2 (difference in slope-0.007, p=0.88). Average scores at day 2 were approximately the same (treatment intercept effect). FIG. 4 shows the change in average score over time for each treatment group.

TABLE 4
Ocular damage: Results of Generalized Estimating Equations model:
Treatment effect describes differences from the control group

| | | Estimate | SE | p-value |
|---|---|---|---|---|
| Intercept (day 2) | | 3.87 | 5.19 | <0.0001 |
| Treatment Effect | Saline | 0.00 | — | — |
| (day 2) | 16 week Human AF | −0.690 | 0.700 | 0.32 |
| | 36 week Human AF | −0.632 | 0.661 | 0.34 |
| Time, units = day | | 0.036 | 0.038 | 0.34 |
| Treatment Time | Saline | 0.00 | — | — |
| | 16 week Human AF | −0.127 | 0.049 | 0.009 |
| | 36 week Human AF | −0.134 | 0.054 | 0.012 |

### Histology

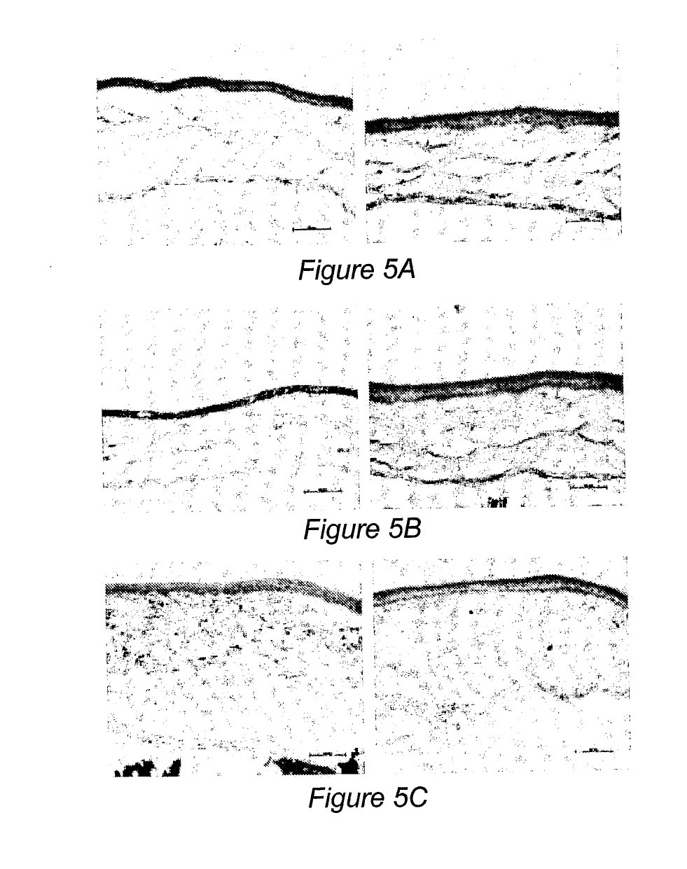

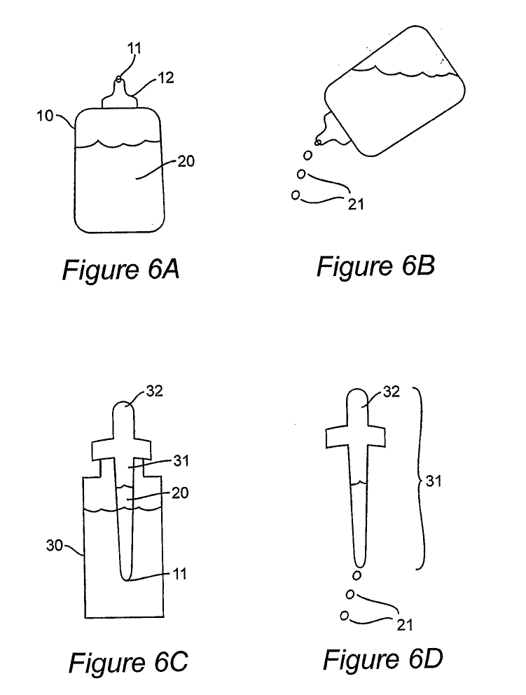

Overall, the organization of the epithelial cell layer and stromal lamellae in the human AF treated corneas was very similar to those of the normal, non-burned, contralateral eye (FIG. 5A-C). This was consistent with the clarity observed macroscopically in most of the eyes of these groups following treatment. Saline solution treated corneas showed increased thickness, inflammatory cells and blood vessels when compared to the non-burned and human AF treated corneas.

### Discussion

Ocular chemical burns trigger a series of events related to a disorganized wound repair.³⁷ It has been reported that alkali burns may produce denaturation of the anterior layers of the cornea, including the epithelium and anterior stroma.³⁸ In an intact cornea, the stroma is mainly composed of fibrillar collagen (types I, III and V), which are distributed in a very organized fashion, contributing to corneal transparency.^{37,39} Keratocytes, which under normal conditions are relatively inactive, are capable of a wide variety of fibroblastic activity following stromal injury.^{37,39} The new collagen produced in these cases is disorganized and can lead to the formation of a corneal scar and neovascularization.^{39,40}

HAM has been used since the early 1940's for the management of ocular surface damage.^{41,42} Several potential mechanisms of action are described by Dua et al.¹ When applied as a biological bandage, HAM reduces discomfort and pain caused by ocular surface damage.^{1,2,5,43} HAM has been used as a substrate for epithelial growth in the management of chemical burns,^{44,45} and has been shown to promote re-epithelialization of ocular surface disorders.^{2,3,5,46} Based on prior work we hypothesize that the corneal epithelial proliferation observed with HAM is due to a humoral process rather than solely the result of mechanical protection afforded by the membrane.¹³ The PEDF present in HAM has shown some ability to activate epithelial cell proliferation in vitro.¹³ HAM has also been shown to reduce inflammation and prevent corneal scarring after ocular burns,³ with clinical evidence that it reduces surface inflammation after chemical eye injuries.² Several antiangiogenic and anti-inflammatory components of HAM have been identified.⁴² Kobayashi, et al.⁴⁷ have also demonstrated that amniotic cell culture supernatant contains potent inhibitors of neovascularization.

In the present study of ocular chemical burn, topical pre-term and term human AF were effective in the reduction of corneal opacity, scarring, and promotion of re-epithelialization when compared to topical isotonic saline. While, it is known that changes in human AF composition occur with advancing pregnancy,^{48,49} no statistically significant differences between pre-term and term human AF were noted in this study.

The percentage of score change was the most consistent method for the assessment of the treatment effect. Variation in the chemical burn scores on day 2 were observed in mice of the same group, perhaps due to individual response to the chemical insult. In this series, the improvement observed between day 2 and day 7, in human AF treated eyes, was significant as compared to controls. Differences noted from day 7 to 14 were less and did not reach significance. This suggests that the treatment benefit is largely conveyed during the first week of treatment.

Previous reports have shown that corneal levels of interleukin (IL) 1α, IL-1β, and IL-6 are elevated in inflammatory conditions, such as chemical burns.^{50,51} Sotozono et al.⁵⁰ demonstrated that IL-1α and IL-6 levels in the cornea are markedly elevated in the regenerated epithelium during the early stages of alkali burn. They suggested that these cytokines may play an important role in the associated corneal damage and repair.⁵¹ Further characterization of components present in the human AF may identify the presence of specific inhibitory cytokines that regulate the wound healing response.

Studies report similarities between HAM and human AF in terms of the balance of pro- and anti-inflammatory cytokines. There are reports showing that human AF has special properties that minimize contraction of the wound, inhibiting various processes that ultimately cause scar.^{27,29,52} Topical delivery of human AF in the form of eye drops has the advantage of avoiding the surgical procedure required with HAM. In addition, the repeated application of preserved human AF may result in a sustained level of beneficial factors rather than a decay in concentration, resulting from elution in HAM. Eventually, human AF may require significantly less processing than HAM, and the storage may be easier. The ability of nonsurgical ophthalmologists to administer the therapy may also improve patient access to treatment.

In some cases the protective mechanical properties of HAM may be desirable. Using the human AF as an option, it may be possible for a bandage collagen contact lens to serve this function. It is conceivable that a collagen contact lens could absorb, concentrate and slowly release the beneficial factors contained in human AF and reduce the need for frequent instillation.

This Example shows that topical application of pre-term and term human AF is an effective therapy for treatment of acute chemical burns of the eye.

### REFERENCES FOR EXAMPLE 1

1. Dua H S, Gomes J A, King A J, Maharajan V S. The amniotic membrane in opthalmology. Surv Ophthahnol 2004; 49:51-77.
2. Ucakhan O O , Koklu G, Firat E. Non-preserved human amniotic membrane transplantation in acute and chronic chemical eye injuries. Cornea 2002; 21:169-172.
3. Meller D, Pires R T F, Mack R J S, et al. Amniotic membrane transplantation for acute chemical or thermal burns. Opthalmology 2000; 107:980-990.
4. Katircioglu Y A, Budak K, Salvarli S, Duman S. Amniotic membrane transplantation to reconstruct the conjunctival surface in cases of chemical burn. Jpn J Opthalmol 2003; 47:519-522.
5. Kobayashi A, Shirao Y, Yoshita T, et al. Temporary amniotic membrane patching for acute chemical burns. Eye 2003; 17:149-158.
6. Yeh L K, Chen W L, Li W, Espana E M, Ouyang J, Kawakita T, Kao W W, Tseng S C, Liu C Y. Soluble lumican glycoprotein purified from human amniotic membrane promotes corneal epithelial wound healing. Invest Opthalmol Vis Sci 2005; 46:479-486.
7. Shao C, Sima J, Zhang S X, Jin J, Reinach P, Wang Z, Ma J X. Suppression of corneal neovascularization by PEDF release from human amniotic membranes. Invest Opthalmol Vis Sci 2004; 45:1758-1762.
8. Kurpakus-Wheater M. Laminin-5 is a component of preserved amniotic membrane. Curr Eye Res 2001; 22:353-357.
9. Keelan J A, Sato T, Mitchell M D. Interleukin (IL)-6 and IL-8 production by human amnion: regulation by cytokines, growth factors, glucocorticoids, phorbol esters and bacterial lipopolyssacharide. Biol Reprod 1997; 57:1438-1444.
10. Ishihara O, Saitoh M, Kinoshita K. Friegen I I improves the reliability of measurement of interleukin-1 related substances in amniotic fluid. Acta Obstet Gynecol Scand 1999; 78:321-325.
11. Dudley D J, Hunter C, Mitchell M D, Varner M W. Amniotic fluid interleukin-10 (IL-10) concentrations during pregnancy and with labor. J Reprod Immunol 1997; 33:147-156.
12. Fukuda H, Masuzaki H, Ishimaru T. Interleukin-6 and interleukin-1 receptor antagonist in amniotic fluid and cord blood in patients with pre-term, premature rupture of the membranes. Int J Gynaecol Obstet 2002; 77:123-129.
13. Shao C, Sima J, Zhang S X, et al. Suppression of corneal neovascularization by PEDF release from human amniotic membranes. Invest Opthalmol Vis Sci 2004; 45:1758-1762.
14. Dawson D W, Volpert O V, Gillis P, et al. Pigment epithelium-derived factor: a potent inhibitor of angiogenesis. Science 1999; 285:245-248.
15. Duh E J, Yang H S, Suzuma I, et al. Pigment epithelium-derived factor suppresses ischemia-induced retinal neovascularization and VEGF-induced migration and growth. Invest Opthalmol Vis Sci 2002; 43:821-829.
16. Duh E J, Yang H S, Haller J A, et al. Vitreous levels of pigment epithelium-derived factor and vascular endothelial growth factor: implications for ocular angiogenesis. Am J Opthalmol 2004; 137:668-674.
17. Shao C, Sima J, Zhang S X, Jin J, Reinach P, Wang Z, Ma J X. Suppression of corneal neovascularization by PEDF release from human amniotic membranes. Invest Opthalmol Vis Sci 2004; 45:1758-1762.
18. Leen M M, Moster M R, Katz L J, Terebuh A K, Schmidt C M, Spaeth G L. Management of overfiltering and leaking blebs with autologous blood injection. Arch Opthalmol 1995; 113:1050-1055.
19. Burnstein A, WuDunn D, Ishii Y, Jonescu-Cuypers C, Cantor L B. Autologous blood injection for late-onset filtering bleb leak. Am J Opthalmol 2001; 132:36-40.
20. Ferreira de Souza R, Kruse F E, Seitz B. [Autologous serum for otherwise therapy resistant corneal epithelial defects—Prospective report on the first 70 eyes]. Klin Monatsbl Augenheilkd 2001; 218:720-726.
21. Goto E, Shimmura S, Shimazaki J, Tsubota K. Treatment of superior limbic keratoconjunctivitis by application of autologous serum. Cornea 2001; 20:807-810.
22. Tananuvat N, Daniell M, Sullivan L J, et al. Controlled study of the use of autologous serum in dry eye patients. Cornea 2001; 20:802-806.
23. Tsubota K, Goto E, Fujita H, Ono M, Inoue H, Saito I, Shimmura S. Treatment of dry eye by autologous serum application in Sjogren's syndrome. Br J Opthalmol 1999; 83:390-395.
24. Zhang Q, Shimoya K, Moriyama A, Yamanaka K, Nakajima A, Nobunaga T, Koyama M, Azuma C, Murata Y. Production of secretory leukocyte protease inhibitor by human amniotic membranes and regulation of its concentration in amniotic fluid. Mol Hum Reprod 2001; 7:573-579.
25. Longaker M T, Adzick N S, Hall J L, et al. Studies in fetal wound healing, VII. Fetal wound healing may be modulated by hyaluronic acid stimulating activity in amniotic fluid. J Pediatr Surg 1990; 25:430-433.
26. Lee H S, Kim J C. Effect of amniotic fluid in corneal sensitivity and nerve regeneration after excimer laser ablation. Cornea 1996; 15:517-524.
27. Longaker M T, Whitby D J, Ferguson M W, Lorenz H P, Harrison M R, Adzick N S. Adult skin wounds in the fetal environment heal with scar formation. Ann Surg 1994; 219:65-72.
28. Gao X, Devoe L D, Given K S. Effects of amniotic fluid on proteases: a possible role of amniotic fluid in fetal wound healing. Ann Plast Surg 1994; 33:128-134.
29. Ozgenel G Y, Filiz G. Effects of human amniotic fluid on peripheral nerve scarring and regeneration in rats. J Neurosurg 2003; 98:371-377.
30. al-Qattan M M, Posnick J C, Lin K Y. The in vivo response of fetal tendons to sutures. Hand Surg [Br] 1995; 20:314-318.
31. Ambati B K, Joussen A M, Ambati J, et al. Angiostatin inhibits and regresses corneal neovascularization. Arch Opthalmol 2002; 120:1063-1068.
32. Sotozono C, He J, Tei M, Honma Y, Kinoshita S. Effect of metalloproteinase inhibitor on corneal cytokine expression after Alkali Injury. Invest Opthalmol Vis Sci 1999; 40:2430-2434.
33. Roper-Hall M J. Thermal and chemical burns. Trans Opthalmol Soc UK 1965; 85:631-653.
34. Seber G A F, Lee A J. Linear Regression Analysis, Hoboken, N.J.: John Wiley, 2003. p. 582.
35. Appendix E: Calculation of the Intraclass Correlation Coefficient. In: Szklo M, Nieto J, eds. Epidemiology: Beyond the Basics. Gaithersburg, Md.: Aspen Publishers; 2000:479-481.
36. Liang K Y, Zeger S L. Longitudinal Data Analysis using Generalized Linear Models. Biometrics 1986; 73:13-22.
37. Wagoner M D. Chemical injuries of the eye: current concepts in pathophysiology and therapy. Surv Opthalmol 1997; 41:275-313.
38. Chuck R S, Behrens A, Wellik S, Liaw L L, Dolorico A M, Sweet P, Chao L C, Osann K E, McDonnell P J, Berns M W. Re-epithelialization in cornea organ culture after chemical burns and excimer laser treatment. Arch Opthalmol 2001; 119:1637-1642.
39. Cintron C, Hong B S, Covington H I. Quantitative analysis of collagen from normal developing corneas and corneal scars. Curr Eye Res 1981; 1:1-8.
40. Chung J H, Fagerholm P. Stromal reaction and repair after corneal alkali wound in the rabbit: a quantitative microradiographic study. Exp Eye Res 1987; 45:227-237.
41. De Rotth A. Plastic repair of conjunctival defects with fetal membranes. Arch Opthalmol 1940; 23:522-525.
42. Sorsby A, Symmons H M. Amniotic membrane grafts in caustic burns of the eye (burns of second degree). Br J Opthalmol 1946; 30:337-345.
43. Hao Y, Ma D H, Hwang D G. Identification of antiangiogenic and antiinflamatory proteins in human amniotic membrane. Cornea 2000; 19:348-352.
44. Shimazaki J, Yang H Y, Tsubota K. Amniotic membrane transplantation for ocular surface reconstruction in patients with chemical and thermal burns. Opthalmology 1997; 104:2068-2076.
45. Azuara-Blanco A, Pillai C T, Dua H S. Amniotic membrane transplantation for ocular surface reconstruction. Br J Opthalmol 1999; 83:399-402.
46. Lee S H, Tseng S C. Amniotic membrane transplantation for persistent epithelial defects with ulceration. Am J Opthalmol 1997; 123:303-312.
47. Kobayashi N, Kabuyama Y, Sasaki S, Kato K, Homma Y. Suppression of corneal neovascularization by culture supernatant of human amniotic cells. Cornea 2002; 21:62-67.
48. Das S K, Foster H W, Adhikary P K, Mody B B, Bhattacharyya D K. Gestational variation of fatty acid composition of human amniotic fluid lipids. Obstet Gynecol 1975; 45:425-432.
49. Arvidson G, Ekelund H, Astedt B. Phospholipid composition of human amniotic fluid during gestation and at term. Acta Obstet Gynecol Scand 1972; 51:71-75.
50. Sotozono C, Jiucheng H, Matsumoto Y, Kita M, Imanishi J, Kinoshita S. Cytokine expression in the alkali-burned cornea. Curr Eye Res 1997; 16:670-676.
51. Becker J, Salla S, Dohmen U, Redbrake C, Reim M. Explorative study of interleukin levels in the human cornea. Graefes Arch Clin Exp Ophthahnol 1995; 233:766-771.
52. Ledbetter M S, Morykwas M J, Ditesheim J A, Vander Ark W D, La Rosee J R, Argenta L C. The effects of partial and total amniotic fluid exclusion on excisional fetal rabbit wounds. Ann Plast Surg 1991; 27:139-145.

### Example 2

### Inhibition of Induced Corneal Neovascularization

Angiogenesis relates to the formation of new blood vessels from pre-existing vascular structures. It is an important pathogenic process in inflammatory and immunologic conditions involving the cornea.

Human amniotic membrane (HAM) is a complex biological tissue that has been used for many years in the management of ocular surface pathology. It is believed to possess anti-angiogenic, epitheliotrophic, and anti-inflammatory properties.¹ The anti-angiogenic protein pigment epithelium derived factor (PEDF), has also been found in HAM.² PEDF is anti-angiogenic in animals models of retinal and corneal neovascularization (NV). In vivo, HAM is bathed with amniotic fluid (human AF). Most of the proteins present in HAM are also found in human AF, including PEDF.

This study compared the efficacy of human AF versus PEDF in the inhibition of corneal NV using a corneal micropocket model in mice.

### Methods

The study protocol was approved by the Johns Hopkins University Animal Care and Use Committee, and human AF was obtained after approval of the Institutional Review Board of the Johns Hopkins University. All animals were treated in observance of the ARVO Statement for the Use of Animals in Ophthalmic and Vision Research.

Five groups of C57BL/6 mice (n−25) were surgically implanted with a hydron polymer-based pellet into a corneal micropocket, using a modification of the technique described by Kenyon, et al.³ The pellet contained either 20 ng of bFGF and 35 μg of sucralfate (Group I [n=5], II [n=6], III [n=4], and IV [n=5]), or 20 ng of bFGF/200 ng of PEDF, and 35 μg of sucralfate (Group V [n=5]). Further, we assigned topical treatment to the first three groups. Group I received pre-term human AF, group II term human AF, and group III isotonic saline (Table 5). Five microliters of the respective solution was applied 5 times a day for 6 days.

TABLE 1
Group Distribution According to Pellet and Topical Treatment

| GROUP | PELLET IMPLANTED | TOPICAL TREATMENT |
|---|---|---|
| I | bFGF | Pre-term Human AF |
| II | bFGF | Term Human AF |
| III | bFGF | Isotonic Saline |
| IV | bFGF | None |
| V | bFGF/PEDF | None |

Eyes were photographed immediately after pellet implantation, and also on postoperative day 6, using a digital camera (Nikon Coolpix 990, Nikon Inc., Melville, N.Y.) with a 17× macro lens attached. Two parameters were used to assess corneal NV: the maximal vessel length (VL) extending from the limbal vasculature towards the pellet, and the contiguous circumferential zone of NV (clock hours of NV, where 1 clock hour equals 30 degrees of arc). The area of NV in mm² (A) was then calculated as described by Kenyon, et al.⁴

A(mm²)=0.2×χ×VL(mm)×CN(mm)

### Statistical Analysis

Descriptive statistics were expressed as mean and standard deviation (SD). Non-parametric tests were used for the analysis. Multiple comparisons (Table 6) were made between the different groups. P<0.01 was considered significant. In order to minimize the type I error introduced with multiple comparisons, Tukey's honestly significant difference (HSD) was used. This test requires the calculation of a minimum significant difference (MSD). MSD is a function of the studentized range statistic, q, and requires a confidence level (a), which was set to 0.01. The mean for each group was compared to the MSD, resulting in a mean difference (MD). When the MD is greater than the calculated MSD, the difference between the two groups compared is significant (p<0.01).

TABLE 6
Group Comparisons

Topical pre-term Human AF (group I) vs. Topical Saline (group III)
Topical term Human AF (group II) vs. Topical Saline (group III)
FGF pellets (group IV) vs. FGF/PEDF pellets (group V)
Topical pre-term Human AF (group I) vs. FGF/PEDF pellets (group V)
Topical term Human AF (group II) vs. FGF/PEDF pellets (group V)
Topical pre-term Human AF (group I) vs. topical term Human
AF (group II)

### Results

The results are presented in Table 7. As can be seen, the area of corneal NV was significantly reduced when comparing each of the human AF treated groups to the saline treated mice (MD=5.35 for the pre-term human AF group, MD=5.05 for the term human AF treated mice). However, no significant difference was found when comparing both human AF groups (MD=0.30).

TABLE 7
Area of Corneal NV on Post-operative Day 6; Descriptive Statistics
are Expressed as Mean Area (mm²) and Standard Deviation (SD)

| Area of | | | | | |
|---|---|---|---|---|---|
| Corneal NV | Group I | Group II | Group III | Group IV | Group V |
| Mean | 1.03 mm² | 1.33 mm² | 6.38 mm² | 6.22 mm² | 1.68 mm² |
| SD | 0.64 | 0.74 | 0.82 | 3.28 | 1.10 |

The MSD was calculated to be 3.72 when a = 0.01.

Consistent with previous observations, implantation of bFGF pellets produced a vigorous neovascular response in all eyes.³ Addition of PEDF to the pellet resulted in a reduced area of NV which was significantly smaller than the area induced by bFGF pellets alone (MD=4.54).

When comparing the human AF treated mice (groups II and III) to the combined bFGF/PEDF pellet group (group V), no significant difference was found in the resulting area of corneal NV. (MD=0.65 for the pre-term human AF group, MD=0.35 for the term human AF treated group).

### Discussion

Under normal conditions, the cornea is an avascular structure. Angiogenic and anti-angiogenic factors are under a delicate balance. PEDF is one of the many anti-angiogenic molecules that has been investigated. It has been shown to inhibit corneal NV in the rat cornea, and migration of endothelial cells in vitro.⁵ Human AF is secreted from a single layer of columnar epithelial cells on the HAM, and thus has shown wound healing and growth factors similar to those found in HAM. Further, it has been demonstrated that human AF contains a significant amount of PEDF.² In this study, both pre-term and term topical human AF appeared to be as effective as PEDF in the inhibition of bFGF-induced corneal NV. Human AF thus provides an alternative in the treatment of various corneal neovascular disorders.

### REFERENCES FOR EXAMPLE 2

1. Dua H S, Gomes J A, King A J, Maharajan V S. The amniotic membrane in opthalmology. Surv Opthalmol 2004; 49:51-77.
2. Shao C, Sima J, Zhang S X, Jin J, Reinach P, Wang Z, Ma J X. Suppression of corneal neovascularization by PEDF release from human amniotic membranes. Invest Opthalmol Vis Sci 2004; 45:1758-1762.
3. Kenyon B M, Voest E E, Chen C C, Flynn E, Folkman J, D'Amato R J. A model of Angiogenesis in the Mouse Cornea. Invest Ophthalnol Vis Sci 1996; 37:1625-1632.
4. Kenyon B M, Browne F, D'Amato R J. Effects of Thalidomide and Related Metabolites in a Mouse Corneal Model of Neovascularization. Exp Eye Res 1997; 64:971-978.
5. Dawson D W, Volpert O V, Gillis P, et al. Pigment Epithelium-Derived Factor: A Potent Inhibitor of Angiogenesis. Science 1999; 285:245-248

### Example 3

### Treatment of Dry Eye Syndrome with Human Amniotic Fluid

### Clinical Results

One patient was enrolled in a clinical protocol in Caracas, Venezuela, with a diagnosis of severe dry eye due to a medical condition named Sjögren's Syndrome. This is a chronic condition associated with dry eye and dry mouth.

This particular patient has been treated in the past with several medications for dry eye, without much success. The patient has a long list of topical medications that have been attempted, with transient/minimal improvement over the years. The frequency of lubricant eye drops has been used by some clinicians as an indicator of disease severity and, at the same time, as a predictor of clinical success of therapy.

The patient started treatment with topical human amniotic fluid (after appropriate serology and sterility conditions of preparation) and after one week, the subjective and objective improvement was impressive. The drops were very well tolerated, no adverse reactions were reported by the patient. The individual reported a very “refreshing” sensation after instillation of the human AF, with improvement in symptoms in a very short period after application. The dosage used in this patient was one drop 4 times a day.

The OSDI is a classification used for subjective assessment of severity of dry eye. There was an important reduction in severity score as revealed in Table 8 below. Similarly, the objective signs at the physical examination improved consistently over time: Oxford classification of epithelial corneal/conjunctival damage decreased, tear production increased slightly in the Schirmer's test, a reduction in the dependence of lubricants was also considerable and most importantly, the visual acuity improved dramatically (see Table 8).

TABLE 8
Results of clinical treatment of dry eye with human AF

| | | 2 Days of | 1 week of | 2 weeks of |
|---|---|---|---|---|
| | Pre-Treatment | Treatment | Treatment | Treatment |
| OSDI | 90 | 72.5 | 37.5 | 27.8 |
| Assessment | | | | |
| Oxford | 4 OU | 4 OU | 3 OU | 2 OU |
| Schirmer 1 test | OD: 0.5 mm | OD: 1 mm | OD: 2 mm | OD: 3 mm |
| | OS: 1 mm | OS: 1 mm | OS: 2 mm | OS: 3 mm |
| Use of | 14 times a day | 14 times a day | 9 times a | 6 times a |
| Artificial Tears | | | day | day |
| (Genteal) | | | | |
| Visual Acuity | 20/150 | 20/150 | 20/60 | 20/40 |

Further, after one week of treatment, examination of the patient showed considerably less conjunctival redness, with a more regular ocular surface and less staining with fluorescein in the cornea (not shown).

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method for treating a disorder or injury in an eye, comprising the steps of
administering amniotic fluid free of amniotic membrane particulate matter to said eye in a quantity sufficient to ameliorate symptoms associated with said disorder or said injury.

2. The method of claim 1, wherein said injury is a chemical burn.

3. The method of claim 1, wherein said disorder is dry eye.

4. The method of claim 1, wherein said disorder is a corneal neovascular disorder.

5. The method of claim 1, wherein said disorder is surface inflammation or intraocular inflammation.

6. The method of claim 1, wherein said disorder is corneal opacity.

7. The method of claim 1, wherein said amniotic fluid free of amniotic membrane particulate matter is human amniotic fluid.

8. The method of claim 1, wherein said amniotic fluid free of amniotic membrane particulate matter is in the form of eyedrops.

9. The method of claim 1, wherein said amniotic fluid free of amniotic membrane particulate matter is released from a collagen contact lens.

10. The method of claim 1, wherein said amniotic fluid free of amniotic membrane particulate matter has been lyophilized and reconstituted.

11. A device and medicament combination for treating a disorder or injury to the eye, comprising
a housing having a reservoir and an orifice for dispensing selected volumes of fluid medicament, wherein said reservoir is operatively connected to said orifice so as to allow said selected volumes to be dispensed through said orifice; and
a fluid medicament which is or contains amniotic fluid free of amniotic membrane particulate matter positioned in said reservoir of said housing.

12. The device and medicament combination of claim 11, wherein said injury is a chemical burn.

13. The device and medicament combination of claim 11, wherein said disorder is dry eye.

14. The device and medicament combination of claim 11, wherein said disorder is a corneal neovascular disorder.

15. The device and medicament combination of claim 11, wherein said amniotic fluid free of amniotic membrane particulate matter is human amniotic fluid.

16. The device and medicament combination of claim 11, wherein said device dispenses eye drops.

17. The device and medicament combination of claim 11, wherein said device dispenses a spray.

18. A device and medicament combination for treating a disorder or injury to the eye, comprising
a housing having a reservoir and an orifice for dispensing selected volumes of fluid medicament, wherein said reservoir is operatively connected to said orifice so as to allow said selected volumes to be dispensed through said orifice; and
a fluid medicament which is or contains amniotic fluid that has the properties of amniotic fluid that has been centrifuged at 1800 rpm positioned in said reservoir of said housing.
